(19) United States
(12) Patent Application Publication
(10) Pub. No.: US 2025/0122140 A1
(43) Pub. Date: Apr. 17, 2025

(54) NOVEL CYCLIC COMPOUNDS, AND USE THEREOF AS PERFUMES

(71) Applicant: MILTITZ AROMATICS GMBH, BITTERFELD-WOLFEN (DE)

(72) Inventors: STEFAN MUELLER, LEIPZIG (DE); ANDREAS PETRI, LEIPZIG (DE); VLADIMIR PRISYAZHNYUK, POCKAU-LENGENFELD (DE)

(73) Assignee: MILTITZ AROMATICS GMBH, BITTERFELD-WOLFEN (DE)

(21) Appl. No.: 18/687,310

(22) PCT Filed: Aug. 25, 2022
(86) PCT No.: PCT/EP2022/073644

§ 371 Date: Feb. 28, 2024

(30) Foreign Application Priority Data
Sep. 1, 2021 (EP) 21194374.1

(51) Int. Cl. C07C 47/42; C07C 47/32; C07C 47/36; C07C 47/46; C11B 9/00

(52) U.S. Cl. CPC C07C 47/42; C07C 47/32; C07C 47/36; C07C 47/46; C11B 9/0034

(57) ABSTRACT

A compound of formula (I)

or a compound of formula (II)
wherein, for each of the compound of formula (I) and the compound of formula (II), R¹ denotes a CH₂OH group, R² denotes a CH═O group or a CH₂OH group, and R³ and R⁴, independently of one another, denote a C₁-C₄ alkyl group.

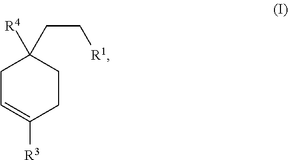

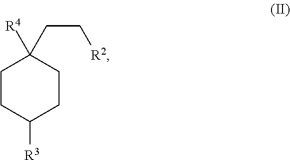

### CROSS REFERENCE TO PRIOR APPLICATIONS

This application is a U.S. National Phase application under 35 U.S.C. § 371 of International Application No. PCT/EP2022/073644, filed on Aug. 25, 2022 and which claims benefit to European Patent Application No. 21194374.1, filed on Sep. 1, 2021. The International Application was published in German on Mar. 9, 2023 as WO 2023/031003 A1 under PCT Article 21(2).

### FIELD

The present invention relates to cyclic compounds and to the use thereof as perfumes.

### BACKGROUND

Cyclic isoprene dimers and applications thereof, including as perfumes, have long been known and are, for example, described in H. Morikawa und S. Kitazume, Ind. Eng. Chem. Prod. Res. Dev., Vol. 18, No. 4 (1979).

GB 1 471 856 A describes the compound 3-(1,4-dimethylcyclohex-3-en-1-yl)propanal as an intermediate in the preparation of various perfumes and synthesis thereof from 1,4-dimethyl-4-vinyl-1-cyclohexene (DMVCH) by hydroformylation with rhodium catalysis.

### SUMMARY

An aspect of the present invention is to develop novel perfumes.

In an embodiment, the present invention provides a compound of formula (I) or a compound of formula (II)

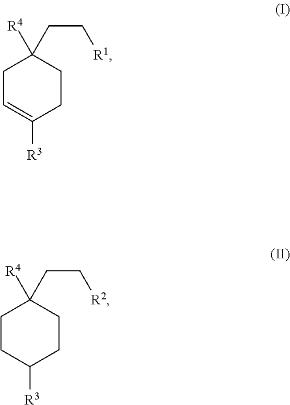

wherein, for each of the compound of formula (I) and the compound of formula (II), R¹ denotes a CH₂OH group, R² denotes a CH═O group or a CH₂OH group, and R³ and R⁴ denote, independently of one another, a C₁-C₄ alkyl group.

Both R³ and R⁴ can, for example, denote a methyl group.

The present invention also relates to the use of a compound of formula (I) or a compound of (II)

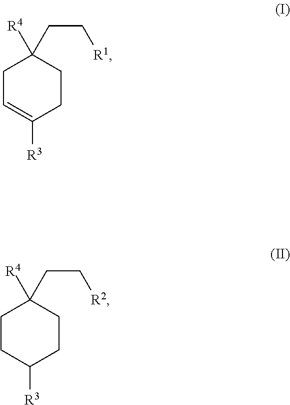

wherein, for each of the compound of formula (I) and the compound of formula (II), R¹ and R² each denote a CH₂OH group or a CH═O group, and R³ and R⁴ denote, independently of one another, a C₁-C₄ alkyl group, as a perfume.

In this case, both R³ and R⁴ can, for example, also denote a methyl group.

### DETAILED DESCRIPTION

The compounds or perfumes of the present invention are all derived from the cyclic isoprene dimer DMVCH, which is synthesized in the manner described in DE 2801910 A1. DMVCH is also available commercially, for example, from the companies Aurora Fine Chemicals, Ambinter, Alfa Chemistry, BOC Sciences and Atomax Chemicals.

In a first step, DMVCH 1 is hydroformylated with rhodium catalysis to obtain 3-(1,4-dimethylcyclohex-3-en-1-yl)propanal 2, which, as stated above, is known as a compound, but not as a perfume.

The compound 3-(1,4-dimethylcyclohexyl)propanal 3 is obtained by hydrogenating the double bond in compound 2 with a supported noble metal catalyst (for example, Pd/C), whereby the compounds 3-(1,4-dimethylcyclohex-3-en-1-yl)propan-1-ol 4 and 3-(1,4-dimethylcyclohexyl)-propan-1-ol 5 are obtained by reducing the aldehyde function in compounds 2 and 3, respectively, (for example, with sodium borohydride).

The synthesis of the compounds 2 to 5 is schematically shown as follows:

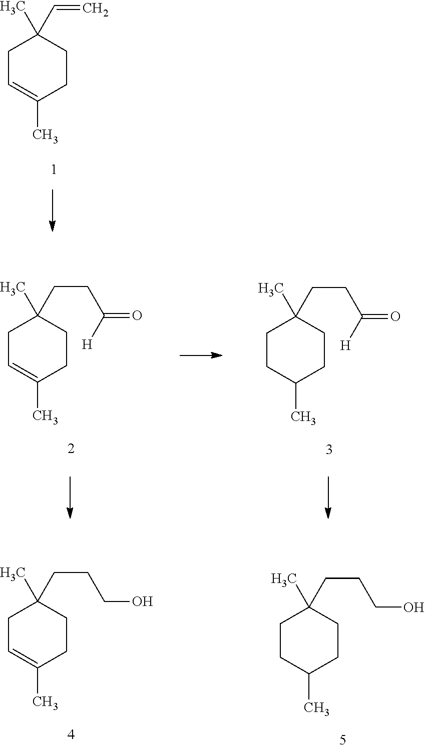

Compounds 3 to 5 and the previously described compound 2 were fully characterized and underwent a sensory assessment by a perfumer. All compounds 2 to 5 show great potential as perfumes.

### Syntheses and Characterizations

### Synthesis of Compound 2

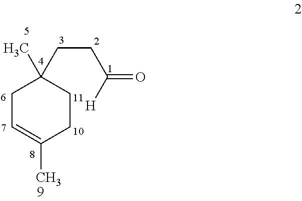

### 3-(1,4-dimethylcyclohex-3-en-1-yl)propanal (Compound 2)

Freshly distilled DMVCH (500 g, 3.67 mol), [Rh] catalyst Rh(H)(CO)(PPh₃)₃ (0.67 g, 0.73 mmol) and triphenylphosphine (0.96 g, 3.67 mmol) were placed in a pressure autoclave. The reaction mixture was hydroformylated with synthesis gas, i.e., CO/H₂ (1:1), at 25 bar and 100° C. for approximately 4 hours.

The crude product contained <1% hydrogenated DMVCH, >90% product. No unreacted DMVCH was detected.

The crude product was distilled at 100° C. under a vacuum of 1 mbar using a packed column. 426 g of Compound 2 was obtained (65.9%).
GC-MS: 166 (M+), 148 (M+-H₂O), 123 (M+-·CH₂CHO), 109 (M+-·CH₂CH₂ CHO), 107 (M+-·CH₂CH₂CHO—CH₃), 68 (100%, ·C₅H₈, retro-Diels-Alder reaction of Me-cyclohexane), 55 (·CH₂CH₂CHO).

NMR:
1H: 0.8 (s, 5-CH₃), 1.3 (t, 11-CH₂), 1.5 (tq, 3-CH₂), 1.6 (s, 9-CH₃), 1.7 (dq, 6-CH₂), 1.8 (tq, 10-CH₂), 2.3 (tt, 2-CH₂), 5.2 (t, 7-H), 9.7 (s, 1-H).
13C: 202 (1-C), 132 (8-C), 119 (7-C), 38 (2-C), 37 (4-C), 33 (6-C), 32 (10-C), 30(3-C), 27 (11-C), 24(9-C), 23 (5-C).

Boiling Point: 70° C. (0.7 mbar)

Density: 0.931 g/cm³ (at 20° C.)

Refractive Index: 1.4773 (at 20° C.)

Sensory Analysis:

The aldehydes of this series of perfumes had strong floral characteristics. The unsaturated aldehyde also had a marked multifaceted green character, with leaf-like and melon rind-like aspects. The aldehyde had a somewhat stronger scent than the corresponding alcohol. The odor profile was relatively constant over 24 hours and remained on the scent strip for at least 7 days.

### Synthesis of Compound 3

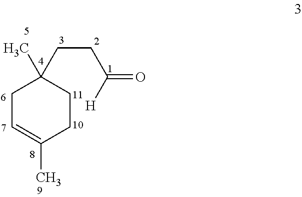

### 3-(1,4-dimethylcyclohexyl)propanal (Compound 3)

Compound 2 (333 g, 2.0 mol) and catalyst 5% Pd/C (3.33 g, 1.6 mmol) were placed in a pressure autoclave. The reaction mixture was hydrogenated with hydrogen at 15 bar pressure and 70° C. for approximately 5 hours.

The crude product was distilled at 75° C. under a vacuum of 1 mbar using a packed column. 240 g of compound 3 was obtained (71.2%).
GC-MS: 168 (M+), 150 (M+-H₂O), 135 (M+-H₂O—CH₃), 124 (M+-·CH₂CHO), 111 (M+-·CH₂CH₂CHO), 95 (·Me-cyclohexane), 81 (·cyclohexane), 69 (100%, ·C₅H₈, retro-Diels-Alder reaction of Me-cyclohexane), 55 (·CH₂CH₂CHO), 41 (allyl, cyclohexane fragmentation).

NMR (mixture of isomers):
1H: 0.7 (s, 5-CH₃), 0.8 (d, 9-CH₃), 1.0 (m, 10-CH₂), 1.1 (m, 7-CH₂), 1.2 (m, 8-H), 1.3 (m, 6-CH₂), 1.4 (m, 11-CH₂), 1.5 (tq, 3-CH₂), 2.3 (tt, 2-CH₂), 9.7 (t, 1-H).
13C: 203 (1-C), 39 (2-C), 37 (4-C), 37 (7-C), 33 (6-C), 32 (10-C), 31 (8-C), 30 (3-C), 27 (10-C), 22 (9-C), 21 (5-C).

Boiling Point: 78° C. (0.7 mbar)

Density: 0.905 g/cm³

Refractive Index: 1.4610 (at 20° C.)

Sensory Analysis:

The saturated aldehyde retained the floral characteristics of this series, but was much greener, reminiscent of watermelon rind, and slightly aggressive. The strength corresponded to that of the unsaturated aldehyde. As with the other perfumes, the odor profile was also very linear over time and remained on the scent strip for at least 7 days.

### Synthesis of Compound 4

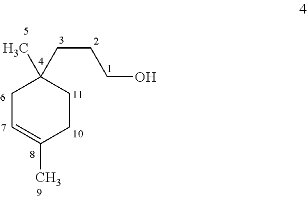

### 3-(1,4-dimethylcyclohex-3-en-1-yl)propan-1-ol (Compound 4)

Compound 2 (300 g, 1.8 mol) was dissolved in 500 ml of ethanol. The reaction mixture was cooled to 0-5° C. Sodium borohydride (75.1 g, 2.0 mol) was added very slowly in portions so that the internal temperature did not exceed 25° C. The reaction time was approximately 4 hours. At the end of the reaction, 400 ml water and 500 ml cyclohexane were added to the reaction mixture. The phases were separated and the organic phase was washed with 100 ml sulfuric acid (5%), and then three times with 100 ml water each time.

The crude product was distilled at 84° C. under a vacuum of 0.3 mbar using a packed column. 145 g of compound 4 was obtained (50.1%).
GC-MS: 168 (M+), 135 (M+-H₂O-·CH₃), 109 (M+-·CH₂CH₂CHO), 68 (100%, ·C₅H₈, retro-Diels-Alder reaction of Me-cyclohexane).

NMR:
1H: 0.8 (s, 5-CH₃), 1.2 (m, 2-CH₂), 1.3 (t, 11-CH₂), 1.5 (tq, 3-CH₂), 1.6 (s, 9-CH₃), 1.7 (dq, 6-CH₂), 1.8 (tq, 10-CH₂), 2.1 (s, —OH), 3.5 (t, 1-CH₂), 5.2 (t, 7-H).
13C: 133 (8-C), 120 (7-C), 64 (1-C), 38 (2-C), 37 (4-C), 34 (6-C), 31 (10-C), 27 (3-C), 27 (11-C), 24 (5-C), 23 (9-C).

Boiling Point: 80° C. (<0.5 mbar)

Density: 0.929 g/cm³ (at 20° C.)

Refractive Index: 1.4873 (at 20° C.)

Sensory Analysis:

The olfactory profile of this unsaturated alcohol was primarily floral, with green and lily-of-the-valley/rose aspects with aqueous/fruity undertones and a sugary/malty sweetness. The odor profile remained largely constant from 0 to 24 hours, and the product remained on the scent strip for more than 7 days. The strength can be considered medium to strong.

### Synthesis of Compound 5

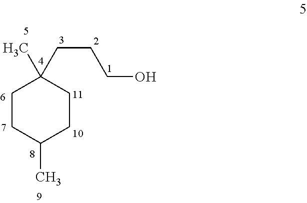

### 3-(1,4-dimethylcyclohexyl)-propan-1-ol (Compound 5)

Compound 3 (168 g, 1.0 mol) was dissolved in 200 ml of ethanol. The reaction mixture was cooled to 0-5° C. Sodium borohydride (41.61 g, 1.1 mol) was added very slowly in portions so that the internal temperature did not exceed 25° C. The reaction time was approximately 4 hours. At the end of the reaction, 200 ml water was added. The phases were separated and the organic phase was diluted with 250 ml cyclohexane and washed with 55 ml sulfuric acid (5%), and then twice with 100 ml water each time.

The crude product was distilled at 90° C. under a vacuum of 0.3 mbar using a packed column. 140 g of compound 5 was obtained (82.3%).
GC-MS: 170 (M+), 152 (M+-H₂O), 137 (M+-H₂O—CH₃), 111 (M+-·CH₂CH₂CH₂OH), 95 (·Me-cyclohexane), 81 (·cyclohexane), 69 (100%, ·C₅H₈, retro-Diels-Alder reaction of Me-cyclohexane), 55 (·CH₂CH₂CHO), 41 (allyl, cyclohexane fragmentation).

NMR (mixture of isomers):
1H: 0.7 (s, 5-CH₃), 0.8 (d, 9-CH₃), 1.0 (m, 10-CH₂), 1.1 (m, 7-CH₂), 1.2 (m, 8-H), 1.3 (m, 6-CH₂), 1.4 (t, 11-CH₂), 1.4 (m, 3-CH₂), 1.5 (m, 2-CH₂), 2.4 (s, —OH), 3.5 (t, 1-CH₂).
13C: 63 (1-C), 41 (2-C), 38 (4-C), 37 (7-C), 33 (6-C), 32 (10-C), 31 (8-C), 30 (3-C), 27 (10-C), 22 (9-C), 21 (5-C).

Boiling point: 92° C. (<0.5 mbar)

Density: 0.901 g/cm³

Refractive index: 1.4669 (at 20° C.)

Sensory Analysis:

The profile of this saturated alcohol was again primarily floral, but much weaker than in the case of the unsaturated alcohol. The product had a dry, waxy olfactory character. It also remained on the scent strip for at least 7 days.

The present invention is not limited to embodiments described herein; reference should be had to the appended claims.

What is claimed is:

1-4. (canceled)

5: A compound of formula (I) 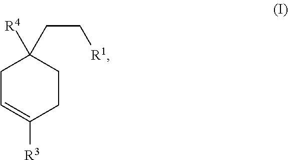
or a compound of formula (II) 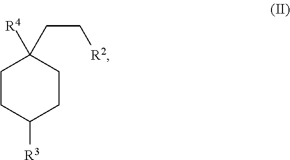
wherein, for each of the compound of formula (I) and the compound of formula (II),
R¹ denotes a CH₂OH group,
R² denotes a CH═O group or a CH₂OH group, and
R³ and R⁴, independently of one another, denote a C₁-C₄ alkyl group.

6: The compound of formula (I) or the compound of formula (II) as recited in claim 5, wherein the C₁-C₄ alkyl group for both R³ and R⁴ is a methyl group.

7: A method of using a compound of formula (I) 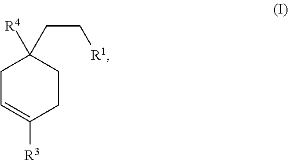
or a compound of formula (II) 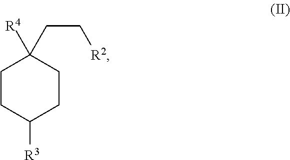
wherein, for each of the compound of formula (I) and the compound of formula (II),
R¹ and R² each denote a CH₂OH group or a CH═O group, and
R³ and R⁴, independently of one another, denote a C₁-C₄ alkyl group,
as a perfume,
the method comprising:
providing the compound of formula (I) or the compound of formula (II); and
using the compound of formula (I) or the compound of formula (II) as a perfume.

8: The method as recited in claim 7, wherein the C₁-C₄ alkyl group for both R³ and R⁴ is a methyl group.
